(19) United States
(12) Patent Application Publication
(10) Pub. No.: US 2012/0082976 A1
(43) Pub. Date: Apr. 5, 2012

(54) METHOD OF SCREENING FOR INSULIN SECRETION-POTENTIATING AGENTS

(75) Inventors: Susumu Seino, Hyogo (JP); Changliang Zhang, Hyogo (JP); Megumi Kato, Hyogo (JP); Tadao Shibazaki, Hyogo (JP)

(73) Assignee: KOBE UNIVERSITY, Kobe-shi (JP)

(21) Appl. No.: 13/264,391

(22) PCT Filed: Apr. 15, 2010
(86) PCT No.: PCT/JP2010/002755

§ 371 Date: Dec. 13, 2011

(30) Foreign Application Priority Data
Apr. 17, 2009 (JP) 2009-100794

(51) Int. Cl. C12Q 1/68; C07K 19/00; C12N 15/85; C12N 5/10; C12N 15/62; C12N 15/63

(52) U.S. Cl. 435/6.1; 536/23.2; 435/320.1; 435/325; 435/365; 530/350

(57) ABSTRACT

Disclosed are a novel method of screening for insulin secretion-potentiating agents as well as means for performing such screening. The means include a DNA encoding fluorescent-labeled Epac2 comprising two different DNAs encoding two different fluorescent proteins which emit fluorescent light with wavelength differing from each other and a DNA encoding Epac2 which are fused together in-frame, and the cells transformed with the DNA. Also disclosed is a method of screening insulin secretion-potentiating agents comprising bringing a candidate compound into contact with cells transformed with the said DNA, and detecting whether the compound binds to Epac2.

### TECHNICAL FIELD

The present invention relates to a method of screening for insulin secretion-potentiating agents, and more specifically to fluorescent-labeled Epac2 gene, cells which are transformed with the gene, and a method of screening for insulin secretion-potentiating agents utilizing such cells.

### BACKGROUND ART

Sulfonylureas (hereinafter referred to as “SU agents”), which are widely used as therapeutic agents for diabetes, are known to exhibit their effects through binding to an SU receptor (SUR1), a component and regulatory subunit of ATP-sensitive K⁺ channels (K_{ATP} channels) (Non-patent documents 1-3). K_{ATP} channels occur on the cell membrane of pancreatic beta-cells and regulate through their opening and closing the amount of insulin to be secreted. Binding of an SU agent to the SU receptors induces closure of the K_{ATP} channels on the surface of pancreatic beta-cells, which brings about depolarization of the cell membrane. This then causes voltage-dependent Ca²⁺ channels to open to allow Ca²⁺ influx into the cells, thereby potentiating insulin secretion. Examples of SU agents include tolbutamide, glibenclamide, chlorpropamide, gliclazide, and the like.

Multiple intracellular signals take part in the mechanism for regulation of insulin secretion. In particular, cAMP occurring in the cell functions as a very important signal molecule for regulation of insulin secretion. It is known that within the mechanism for regulation of insulin secretion caused by cAMP, there are two types of pathways, which are protein kinase A-dependent and independent pathways. Among the protein kinase A-independent pathways, there is known a pathway which is mediated by Epac 2 (exchange protein directly activated by cAMP 2: a guanine-nucleotide-exchanging factor (GEF) for the small G-protein Rapt that is activated by cAMP) (Non-patent documents 4-6). Epac2 activated by direct binding of cAMP has a guanine-nucleotide-exchanging activity for Rap1, and this activity activates Rap1, thereby promoting insulin secretion.

As noted above, while there are a variety of mechanisms for regulation of insulin secretion, the mechanisms of actions of therapeutic agents for diabetes, including SU agents, are yet to be fully elucidated.

There is an isoform of Epac2 structurally similar to it (Epac1) (Non-patent document 7). Epac1 has a cAMP-binding domain, a Disheveled, Egl-10, Plechstrin (DEP) domain, which plays a role in membrane localization of Epac1, a Ras-exchanger motif (REM), and a GEF domain at the carboxy terminus. Epac2, which is structurally similar to Epac1, has in addition to the above domains, a second cAMP-binding domain at its amino-terminus and a Ras-association (RA) domain that interacts with Ras. Both Epac1 and Epac2 take part in intracellular signal transmission via cAMP.

For monitoring signal transmission via cAMP within the cell, a fusion protein has been developed as fluorescence resonance energy transfer (FRET) sensor which monitors Epac1 activation in living cells, in which protein partial or full-length Epac1 is sandwiched between two other proteins which emit fluorescent light with different colors, i.e., cyan and yellow fluorescent proteins (Non-patent documents 8-10).

“FRET” is a phenomenon that when one (donor) of two dye molecules, e.g., fluorescent proteins, located in close proximity with each other is irradiated with light having a certain wavelength (excitation light) to excite the dye molecule, the excitation energy is transferred to the other dye molecules (acceptor) located in close proximity through resonance between electrons. According to this phenomenon, light having a certain wavelength (fluorescent light) is emitted from the acceptor by the energy of light absorbed by the donor. Thus, the FRET technique is a method to detect changes in fluorescence caused by FRET.

When a fusion protein consisting of cyan and yellow fluorescent proteins and partial or full-length Epac1 sandwiched between them (fluorescent-labeled Epac1) is irradiated with excitation light (wavelength: approx. 440 nm) for the cyan fluorescent protein, (which emits fluorescent light with the shorter wavelength), the protein is excited, and FRET taking place with part of this excitation energy, the yellow fluorescent protein is also excited and emits fluorescent light having the wavelength at 535 nm. On the other hand, part of the excitation energy in the cyan fluorescent protein does not cause FRET, but fluorescent light having the wavelength at 480 nm is emitted from the cyan fluorescent protein. Thus, the ratio in intensity between the fluorescent light having two different wavelengths emitted from the fluorescent-labeled Epac1 (fluorescence intensity at the wavelength of 535 nm/fluorescence intensity at the wavelength of 480 nm) is dependent on the conformation and distance between the cyan and yellow fluorescent proteins in the molecule. Therefore, when the conformation of the molecule changes due to some modification or binding with other molecules, the ratio also changes accordingly. As the high-order structure of fluorescent-labeled Epac1, for example, changes when cAMP binds to the molecule, detection of this change by the FRET technique allows detection of whether a cAMP molecule binds to Epac1 or not. Thus, by measuring the change in the intensity of fluorescent light emitted using the FRET technique while irradiating fluorescent-labeled Epac1 with light having the wavelength at 440 nm, presence of signal transmission via Epac1 can be detected.

While a method has been known to measure signal transmission employing Epac1 as a sensor in the FRET technique (FRET sensor) as mentioned above, no method is known in which full-length Epac2 is utilized as a FRET sensor.

Epac2 is known to mediate insulin secretion via a cAMP-dependent, protein kinase A (PKA)-independent pathway (Non-patent documents 11 and 12). Epac2 was originally identified as an interacting molecule (cAMP-GEF II) with SUR1, a regulatory subunit of K_{ATP} channels (Non-patent document 13).

SUR1 is known to be the target molecule of SU agents. Binding of an SU agent to SUR1 induces the K_{ATP} channel closure on the one hand and on the other hand let the voltage-dependent Ca²⁺ channels open, and this, by allowing Ca²⁺ influx into the beta-cells, enhances insulin secretion (Non-patent document 2). Studies of mice lacking Kir6.2 gene, a subunit of K_{ATP} channels, and mice lacking SUR1 gene confirm that closure of the K_{ATP} channels is prerequisite for SU agents to enhance insulin secretion (Non-patent documents 14-16). Thus, it has been thought that it is via K_{ATP} channels that SU agents exhibit their pharmacological effects as therapeutic agents for diabetes.

Recently, mutations in Kir6.2 or SUR1 have been shown to cause neonatal diabetes mellitus with various symptoms due to the impaired function of K_{ATP} channels (Non-patent document 17). Glycemic control was found to be improved in many of such patients when insulin injection was replaced by oral high-dose SU agents (Non-patent document 18).

Further, there is a clinical report that among SU agents, gliclazide was not effective in a patient with neonatal diabetes, while glibenclamide improved glycemic control in such a patient (Non-patent document 19). The finding suggests that the mechanism of action of SU agents is not uniform. Thus, it is essential to know the mechanisms of action of SU agents for providing proper treatment for patients with neonatal diabetes. Furthermore, elucidation of unknown mechanisms of action is important, for it would provide a clue to and means for development of a new way of medical treatment of diabetic patients with diverse background factors, as well as for development of new therapeutic agents for such treatment.

### CITATION LIST

### Non Patent Literature

### [NPL 1]

Seino S., (1999) Annu. Rev. Physiol. 61, 337-62

### [NPL 2]

Henquin J. C., (2000) Diabetes 49, 1751-60

### [NPL 3]

Proks P. et. al., (2002) Diabetes 51, S368-76

### [NPL 4]

de Rooji J. et. al., (1998) Nature 396, 474-7

### [NPL 5]

Kawasaki H. et. al., (1998) Science 282, 2275-9

### [NPL 6]

de Rooji J. et. al., (2000) J. Biol. Chem. 275, 20829-36

### [NPL 7]

Bos J. L. et. al., (2003) Mol. Cell. Biol. 4, 733-8

### [NPL 8]

Nikolaev V. O. et. al., (2004) J. Biol. Chem. 279, 37215-8

### [NPL 9]

Dipilato L. M. et. al., (2004) Proc. Natl. Acad. Sci. USA. 101, 16513-8

### [NPL 10]

Ponsioen B. et. al., (2004) EMBO Rep. 5, 1176-80

### [NPL 11]

Holz G. G. et. al., (2004) Diabetes 53, 5-13

### [NPL 12]

Seino S. et. al., (2005) Physiol. Rev. 85, 1303-42

### [NPL 13]

Ozaki N. et. al., (2000) Nat. Cell. Biol. 2, 805-811

### [NPL 14]

Mild T. et. al., (1998) Proc. Natl. Acad. Sci. USA. 95, 10402-6

### [NPL 15]

Seghers V. et. al., (2000) J. Biol. Chem. 275, 9270-7
[NPL 16] Shiota C. et. al., (2002) J. Biol. Chem. 277, 37176-83

### [NPL 17]

Hattersley A. T. et. al., (2005) Diabetes 54, 2503-13

### [NPL 18]

Pearson E. R. et. al., (2006) N. Engl. J. Med. 355, 467-77

### [NPL 19]

Koster J. C., (2008) J. Clin. Endocrinol. Metab. 93, 1054-61

### SUMMARY OF INVENTION

### Technical Problem

Against the above background, it is an object of the present invention to provide a method of screening for new insulin secretion-potentiating agents which target Epac2, the method utilizing Epac2 fused with two fluorescent proteins as a sensor. It is another object of the present invention to provide a method which is based on their binding to Epac2, of screening of known antidiabetic agents including SU agents, for finding out how they are used properly.

### Solution to Problem

The present inventors found a new mechanism of action of SU agents, which had been known to target the SUR1 molecule, a mechanism in which they act via their binding to Epac2. The present invention was completed on the basis of this finding and through further studies.

Thus the present invention provides what follows.

1. A DNA encoding fluorescent-labeled Epac2 comprising two different DNAs encoding two different fluorescent proteins which emit fluorescent light with wavelength differing from each other and a DNA encoding Epac2 which are fused together in-frame.

2. The DNA according to 1 above, wherein the two different fluorescent proteins are a cyan fluorescent protein and a yellow fluorescent protein.

3. The DNA according to 2 above, wherein the DNA encoding the cyan fluorescent protein and the DNA encoding the yellow fluorescent protein are fused to the 5′- and 3′-terminuses, respectively, of the DNA encoding Epac2.

4. The DNA according to 2 or 3 above, wherein the cyan fluorescent protein is ECFP and the yellow fluorescent protein is EYFP.

5. An expression vector comprising an incorporated DNA according to one of 1 to 4 above.

6. The expression vector according to 5 above, wherein the expression vector is for mammalian cells.

7. A cell which is a transformant carrying the expression vector according to 5 or 6 above.

8. The cell according to 7 above, wherein the cell is a mammalian cell.

9. The cell according to 8 above, wherein the cell does not express SUR1 gene.

10. The cell according to 9 above which is a COS cell.

11. A fluorescent-labeled Epac2 which is a fusion protein comprising Epac2 and two different fluorescent proteins fused thereto, wherein the two different fluorescent proteins emit fluorescent light with wavelength differing from each other.

12. A fluorescent-labeled Epac2 which is a fusion protein comprising two different fluorescent proteins and Epac2, wherein the fusion protein is expressed in the cell according to one of 7 to 10 above.

13. A method of screening for an insulin secretion-potentiating agent comprising the steps of providing candidate compounds for an insulin secretion-potentiating agent, bringing each candidate compound into contact with the cell according one of 7 to 10 above, irradiating the cell with the excitation light for the shorter wavelength fluorescent protein of the two different fluorescent proteins before and after the contact of the cell with the candidate compound to measure the intensity of the fluorescent light emitted from each of the two different fluorescent proteins included in the fluorescent-labeled Epac2 in the cell, detecting changes in the intensity ratio between the two different fluorescent lights before versus after the contact of the candidate compound with the cell, and selecting a candidate compound which caused a change as an insulin secretion-potentiating agent.

14. A method of screening for an insulin secretion-potentiating agent comprising the steps of providing candidate compounds for an insulin secretion-potentiating agent, bringing each candidate compound into contact with the fluorescent-labeled Epac2 according to 11 or 12 above, irradiating the same with the excitation light for the shorter wavelength fluorescent protein of the two different fluorescent proteins before and after the contact of the same with the candidate compound to measure the intensity of the fluorescent light emitted from each of the two different fluorescent proteins included in the fluorescent-labeled Epac2, detecting changes in the intensity ratio between the two different fluorescent lights before versus after the contact with the candidate compound, and selecting a candidate compound which caused a change as an insulin secretion-potentiating agent.

### ADVANTAGEOUS EFFECTS OF INVENTION

The present invention enables to perform screening for insulin secretion-potentiating agents which target Epac2. As no insulin secretion-potentiating agent which targets Epac2 is known so far, the present invention can be used in screening for anti-diabetic agents which work by a novel mechanism of action. Considering that the mechanism of the development of diabetes is not fully understood, and that there are patients who resist current medical treatments, the method according to the present invention is meaningful.

### BRIEF DESCRIPTION OF DRAWINGS

FIG. 1 is a vector map for a wild-type mouse Epac2 expression vector, pFLAG-Epac2.

FIG. 2 is a vector map for a mutant-type mouse Epac2 expression vector, pFLAG-Epac2 G114E G244D.

FIG. 3a is a vector map for pFLAT-CMV-2, and FIG. 3b the nucleotide sequence of its multicloning site.

FIG. 4 shows a gene map for vector pECFP-C1 and the nucleotide sequence of its multicloning site.

FIG. 5 shows a gene map for vector pEYFP-N1 and the nucleotide sequence of its multicloning site.

FIG. 6 is a schematic diagram of the secondary structures of fluorescent-labeled mouse Epac2 and a fluorescent-labeled, mutant-type mouse Epac2: ECFP: cyan fluorescent protein, EYFP: yellow fluorescent protein, A and B: cAMP-binding domain, DEP: [disheveled, Egl-10, Plechstrin domain, REM: Ras-exchanger motif, RA: Ras-association domain, GEF: GEF domain.

FIG. 7 is a graph showing the dynamic analysis using the FRET technique of fluorescent-labeled mouse Epac2 in COS-1 cells in the presence of 8-Bromo-cAMP. The vertical axis indicates R/R₀ values, and the horizontal axis the time that has lapsed since the addition of 8-Bromo-cAMP. Data are shown for (a) fluorescent-labeled mouse Epac2, and (b) a fluorescent-labeled, mutant-type mouse Epac2, respectively.

FIG. 8 is a graph showing the effect of SU agents on the dynamic analysis using the FRET technique of fluorescent-labeled mouse Epac2 in MIN6 cells. The vertical axis indicates R/R₀ values, and the horizontal axis the time that has lapsed since the addition of SU agents. As SU agents, (a) tolbutamide and (b) glibenclamide were added, respectively.

FIG. 9 is a graph showing the effect of SU agents on the dynamic analysis of fluorescent-labeled mouse Epac2 in COS-1 cells using the FRET technique. The vertical axis indicates R/R₀ values, and the horizontal axis the time that has lapsed since the addition of SU agents. As SU agents, (a) tolbutamide, (b) glibenclamide, (c) chlorpropamide, (d) acetohexamide, (e) glipizide, (f) gliclazide, and (g) nateglinide were added, separately.

FIG. 10 is a graph showing the competitive property of the biding of tritium-labeled glibenclamide to mouse Epac2. (a) Black circle: Total binding, Open circle: Binding in the presence of 100 micro M unlabeled glibenclamide. The vertical axis indicates the radioactivity (DPM) of bound tritium-labeled glibenclamide, and the horizontal axis the amount of tritium-labeled glibenclamide which was added. (b) Open circle: unlabeled glibenclamide, Black circle: tolbutamide, Open triangle: 8-Bromo-cAMP. The vertical axis indicates the proportion (%) of bound tritium-labeled glibenclamide, and the horizontal axis the amount (−log M) of the compounds added to for competition.

FIG. 11 shows activation of Rap1 induced by SU agents in MIN6 cells. In each figure, the upper part indicates the amount of GTP-bound Rap1, the lower part the total amount of Rap1. (a) Tolbutamide, (b) glibenclamide, (c) chlorpropamide, (d) acetohexamide, (e) glipizide, and (f) gliclazide were added, respectively.

FIG. 12 shows activation of Rap1 in Epac2-lacking pancreatic beta-cells by SU agents. Represented by the signs in the figure are: TLB: tolbutamide, CLP: chlorpropamide, ACT: acetohexamide, GLP: glipizide, and GLB: glibenclamide, respectively. The data are from (a) Epac2-lacking pancreatic beta-cells, and (b) Epac2-lacking pancreatic beta-cells in which introduced mouse Epac2 is expressed.

FIG. 13 is a graph showing the amount of insulin secreted from the pancreatic beta-cells of wild-type mice (open bars) and Epac2-lacking mice (black bars), respectively. (a) Glucose or KCl, (b) tolbutamide, (c) glibenclamide, and (d) gliclazide were added, respectively.

FIG. 14 is a graph showing serum insulin concentration and blood glucose concentration in wild-type mice (WT) and Epac2-lacking mice (Epac2KO). Open bars and open circles indicate data for wild-type mice, and black bars and black circles for Epac2-lacking mice. (a) Glucose alone was, or (b) glucose and tolbutamide were, administered.

### DESCRIPTION OF EMBODIMENTS

In the present invention, “Epac2”, when simply so referred to, means mammalian Epac2, in particular mouse and human Epac2, and inter alia, wild-type Epac2. The cDNA sequence of mouse wild-type Epac2 is shown as SEQ ID NO:1, and the amino acid sequence as SEQ ID NO:2, respectively. The cDNA sequence of human wild-type Epac2 is shown as SEQ ID NO:3, and the amino acid sequence as SEQ ID NO:4, respectively. Human Epac2 cDNA can be obtained in a publicly known manner (Non-patent document 5).

The amino acid sequences of mouse and human wild-type Epac2 both consist of 1011 amino acids residues, which differ in only 25 amino acids from each other. And in each of five out of those different amino acids, the difference is limited between similar amino acid pairs (one acidic amino acid, one basic amino acid, two branched amino acids, and one hydroxy amino acid). Thus, the homology between them is very high, i.e., not less than 98%. This high homology strongly suggests their substantial equivalence in structure and function. Meanwhile, in the present invention, “Epac2”, when simply so referred to, includes not only the naturally occurring full-length Epac2 but also such peptides as comprising a partial amino acid sequence of Epac2 including its cAMP-binding domain, Disheveled, Egl-10, Plechstrin (DEP) domain, which plays a role in membrane localization, Ras-exchanger motif (REM) domain, GEF domain at the carboxy terminus, cAMP-binding domain at the amino terminus, and Ras-association (RA) domain, which interacts with Ras.

In the present invention, “fluorescent-labeled Epac2” means a protein comprising Epac2 and two different fluorescent proteins which are fused to the former. Although any combination of these two different fluorescent proteins is allowed to be fused to Epac2 insofar as the combination of the fluorescent protein, fused to Epac2, can cause FRET, preferred is a combination of a cyan fluorescent protein and a yellow fluorescent protein, and more preferred is the combination of ECFP (Enhanced Cyan Fluorescence Protein) and EYFP (Enhanced Yellow Fluorescence Protein).

When the combination of fluorescent proteins is employed which consists of a cyan fluorescent protein and a yellow fluorescent protein, though each of those proteins may be fused to Epac2 either on the N-terminus or the C-terminus thereof insofar as the fusion protein thus formed causes FRET, it is preferred that a cyan fluorescent protein is fused on the N-terminus, and a yellow fluorescent protein on the C-terminus.

In the present invention, the term “the FRET technique” means the method in which a change occurring in fluorescent-labeled Epac2 is measured as a change in fluorescent light emitted by FRET. Also in the present invention, the term “fluorescent light” means the fluorescent light emitted from fluorescent-labeled Epac2 by FRET.

Though any expression vectors may be used in the present invention, without any particular limitation, insofar as fluorescent-labeled Epac2 incorporated in it is expressed, expression vectors for mammalian cells are preferred. With the term “for mammalian cells”, it is meant that the vector is capable of working as an expression vector in mammalian cells.

There is no particular limitation as to the cells used in the present invention insofar as fluorescent-labeled Epac2 is expressed in them when they are transformed with the expression vector according to the present invention, and they may be either prokaryotic cells including E. coli, or eukaryotic cells such as yeast or mammalian cells.

When mammalian cells are used in the present invention, there is no particular limitation as to the species from which the cells originate, and cells originating from human, ape, mouse or rat may be preferably used. In addition, there is no particular limitation as to what types of cells may be used, and such cells as fibroblasts, cells originating from kidney, epithelioid cells and pancreatic beta-cells may be used, and further, any of normal cells, cancerated cells, cell lines and primary culture cells may be used.

Furthermore, mammalian cells used in the present invention may be either those cells which express K_{ATP} channels, such as MIN6 cells originating from pancreatic beta-cells, or those cells which do not express K_{ATP} channels, such as COS cells like COS-1 cells. However, in the case where influence of any interaction is to be avoided between SUR1 and fluorescent-labeled Epac2, cells which do not express K_{ATP} channels are preferably chosen. The term “cells which do not express K_{ATP} channels” includes not only those cells which do not express any K_{ATP} channels at all, but also such cells that express K_{ATP} channels but only in too small an amount to exert an influence on the measurements obtained using the FRET technique.

The method of screening for insulin secretion-potentiating agents according to the present invention may be performed by observing the changes in fluorescence using the FRET technique after, or both before and after, the cells expressing the fluorescent-labeled Epac2 are brought into contact with agents. If the fluorescent-labeled Epac2 consists of Epac2 to which a cyan fluorescent protein and a yellow fluorescent protein have been fused, the wavelength of the light that can be used as excitation light in the FRET technique is 435-445 nm, preferably 440 nm. And the fluorescent light emitted in this case has the wavelengths of 475-485 nm and 530-540 nm, in particular 480 nm and 535 nm.

Further, in the method of screening for insulin secretion-potentiating agents according to the present invention, a homogenate of cells expressing fluorescent-labeled Epac2 can also be used, as well as the fluorescent-labeled Epac2, fully or partly purified from the homogenate.

There is no particular limitation as to insulin secretion-potentiating agents which can be screened according to the present invention insofar as they potentiate insulin secretion directly or indirectly via Epac2, and they include agents for treating both type 1 and type 2 diabetes. The term of acting “directly via Epac2” means that an agent acts through direct binding to Epac2, and the term of acting “indirectly via Epac2” means that an insulin secretion-potentiating agent causes signal transmission through its action on a molecule which is associated with Epac2, like acting via proteins upstream of Epac2 in the signal transmission pathway, such as Rab3, or acting via a molecule which binds to Epac2, though the agent itself does not directly bind to Epac2.

The present invention can thus be used to perform screening for insulin secretion-potentiating agents which act via Epac2. This in reverse means that the present invention can be used to perform screening for agents which act not via Epac2 among insulin secretion-potentiating agents including known anti-diabetic drugs.

Further, the present invention can be used in screening for therapeutic agents for diabetic patients lacking K_{ATP} channels or for evaluation of their pharmacological effects. The term “diabetic patients lacking K_{ATP} channels” herein means those diabetic patients who hereditarily lack K_{ATP} channels and includes neonatal diabetic patients who have such a hereditary trait. Therapeutic agents to be screened or evaluated for pharmacological effects herein include, for example, SU agents. The present invention revealed that some SU agents exhibit their pharmacological activity via Epac2, while others not via Epac2. Those SU agents which are expected to work in treating diabetic patients lacking K_{ATP} channels are those which exhibit their pharmacological effect via Epac2, and such agents can be screened, and be evaluated for their effects, using the present invention.

### Examples

The present invention will be described in further detail with reference to examples.

However, it is not intended that the present invention be limited to the examples. In particular, while the examples shown below are on mouse Epac2, the fact that mouse Epac2 and human Epac2, as mentioned, both consist of 1011 amino acids and the homology between them is not less than 98% strongly suggests their substantial equivalency in structure and function. Therefore, all the results obtained in the following examples on mouse Epac2 must be valid also for human Epac2 with substantial equivalency. Meanwhile, all the animal experiments were conducted according to the guidelines of Ethics Committee, School of Medicine, Kobe University.

### Reagents:

Glibenclamide was purchased from ALEXIS (San Diego, USA). Tolbutamide, chlorpropamide, acetohexamide, glipizide, nateglinide and 12-O-tetradecanoyl-phorbol-13-acetate (TPA) were purchased from SIGMA (Saint. Leuis, USA). Gliclazide was purchased from LKT laboratory (Saint Pole, USA). Tritium-labeled glibenclamide was purchased from PerknElmer (Waltham, USA).

### Animals:

Mice lacking Epac2 were prepared by the technique disclosed in Shibasaki T. et al. Proc. Natl. Acad. Sci. USA 104, 19333-9 (2007). Wild-type mouse (C57BL/6) were employed as the control group in all the animal experiments.

### Pancreatic Beta-Cells Lacking Epac2:

Pancreatic beta-cells lacking Epac2 were obtained according to the technique disclosed in Shibasaki T. et al. Proc. Natl. Acad. Sci. USA 104, 19333-9 (2007), by isolating from the mice which were bred after crossing Epac2-lacking mice with IT6 mice, in which SV40 large T antibody is expressed under the control of human insulin gene promoter. The method for preparing IT6 mice is described in Miyazaki J, et al., Endocrinology, 127, 126-132 (1990). Further, MIN6 cells were prepared by the technique disclosed in Japanese patent application publication No. 2002-125661.

### Mouse Epac2 Expression Vector:

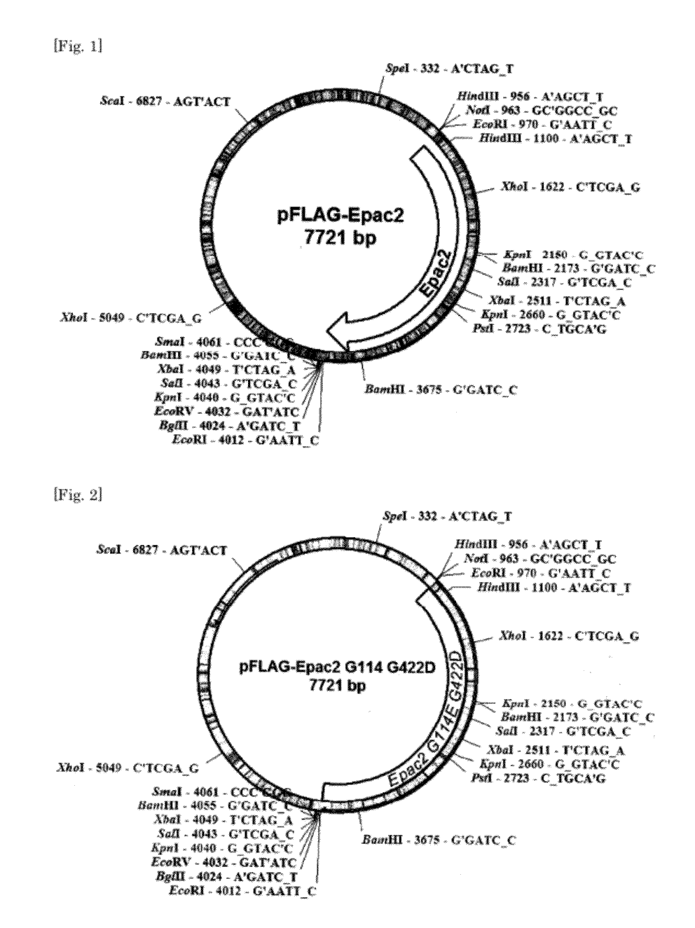

Wild-type mouse Epac2 expression vector (pFLAG-Epac2: FIG. 1) and mutant-type mouse Epac2 expression vector (pFLAG-Epac2 G114E G422D: FIG. 2) employed are disclosed in Ozaki N. et. al., (2000) Nat. Cell. Biol. 2, 805-811 (Non-patent document 13). In the wild-type mouse Epac2 cDNA (SEQ ID NO:1), the first 14 bases and the last 12 bases except the stop codon (tag) are the primer sequences used in the PCR.

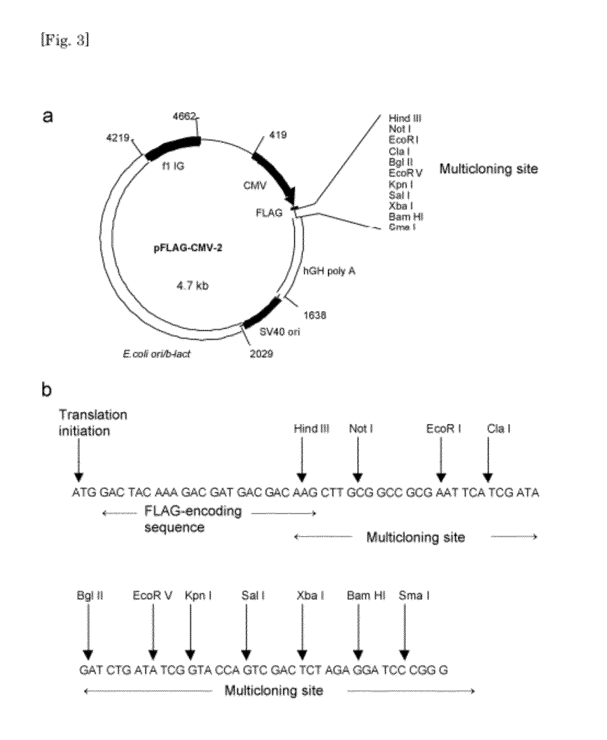

The vector map for pFLAT-CMV-2, which were used for construction of pFLAG-Epac2 and pFLAG-Epac2 G114E G422D, is shown in FIG. 3a, and the nucleotide sequence of its full-length DNA is shown in SEQ ID NO:5, the nucleotide sequence for whose multicloning site is shown in FIG. 3b and as SEQ ID NO:6, respectively. In the expression vectors pFLAG-Epac2 and pFLAG-Epac2 G114E G422D, Epac2 gene is inserted in the EcoRI position of pFLAT-CMV-2 vector.

Further, the cDNA sequence for the mouse mutant-type Epac2 employed above is shown as SEQ ID NO:7, and its amino acid sequence in SEQ ID NO:8, respectively.

Construction of Cyan and yellow Fluorescent-Labeled Mouse Epac2 Expression Vector—Step 1:

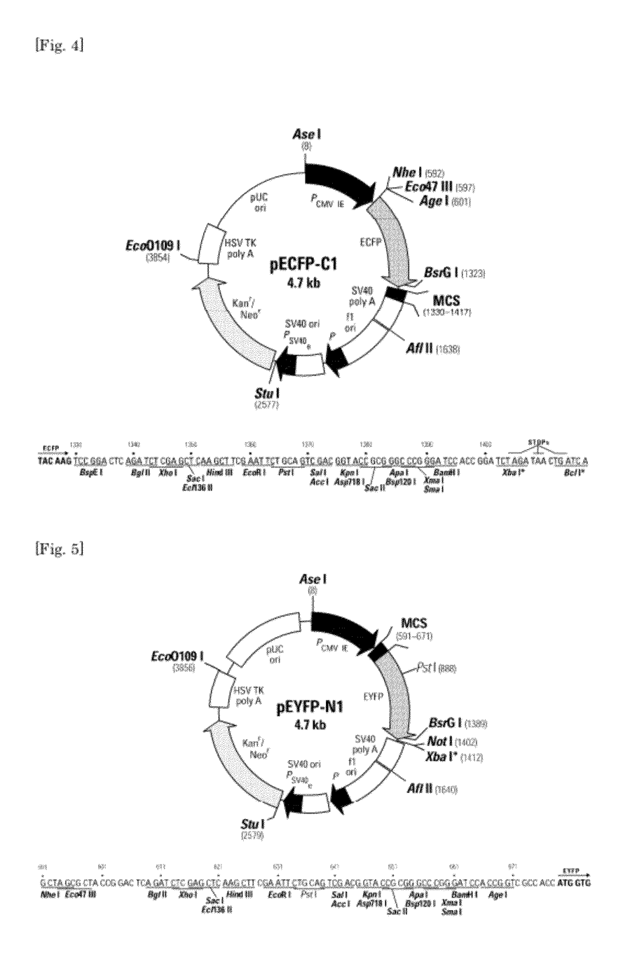

Using the above mouse Epac2 expression vector, pFLAG-Epac2, as a template, PCR was performed with primer BglII-Epac2 (5′-agatctatggtcgctgcgca-3′, SEQ ID NO:9) and primer Epac2-EcoRI (5′-gaattctggccttcgagg-3′: SEQ ID NO:10) to amplify cDNA for mouse Epac2. Using PfuDNA polymerase, the PCR procedure followed consisted of 10 cycles of (95 deg C.: 30 sec, 55 deg C.: 30 sec, 68 deg C.: 2 min), 30 cycles of (94 deg C.: 30 sec, 50 deg C.: 30 sec, 68 deg C.: 3 min) and then the final reaction at 68 deg C. for 5 min. The mouse wild-type Epac2 cDNA (SEQ ID NO:1 as mentioned above) thus amplified was digested with BglII and EcoRI, and then inserted into an expression vector for a cyan fluorescent protein (ECFP), pECFP-C1 (FIG. 4, Clontech), which had been digested with BglII and EcoRI, the DNA sequence for the multicloning site of which is shown in SEQ ID NO:11. This vector thus obtained was named pECFP-mouse Epac2 expression vector. The cDNA sequence for the cyan fluorescent protein (ECFP) is shown in SEQ ID NO:12, and its amino acid sequence in SEQ ID NO:13, respectively.

### Construction of Cyan and Yellow Fluorescent-Labeled Mouse Epac2 Expression Vector—Step 2:

Using an yellow fluorescent protein expression vector, pEYFP-N1 (FIG. 5, Clontech) (the DNA sequence of the multicloning site of this vector is shown in SEQ ID NO:14), as a template, PCR was performed with primer GFP-fw (5′-atggtgagcaagggcg-3′: SEQ ID NO:15) and primer GFP-ry (5′-cttgtacagctcgtccat-3′: SEQ ID NO:16) to amplify the cDNA for the yellow fluorescent protein EYFP. Using PfuDNA polymerase, the PCR procedure followed consisted of 10 cycles of (95 deg C.: 30 sec, 55 deg C.: 30 sec, 72 deg C.: 1 min), then 30 cycles of (94 deg C.: 30 sec, 52 deg C.: 30 sec, 68 deg C.: 1 min) and then the final reaction at 68 deg C. for 5 min. Using the cDNA for EYFP thus obtained by PCR as a template, PCR was performed with a primer EcoRI-EYFP (5′-gaattcatggtgagcaagg-3′: 17) and a primer EYFP-EcoRI (5′-gaattccttgtacagctcgt-3′: SEQ ID NO:18) to amplify EYFP cDNA to which was added an EcoRI site. The cDNA sequence for the yellow fluorescent protein (EYFP) is shown in SEQ ID NO:19, and its amino acid sequence in SEQ ID NO:20, respectively. In the cDNA, the top 13 bases and the rearmost 14 bases except the stop codon “tga” are the primer sequences. Using PfuDNA polymerase, the PCR procedure followed consisted of 10 cycles of (95 deg C.: 30 sec, 55 deg C.: 30 sec, 72 deg C.: 1 min), and 30 cycles of (94 deg C.: 30 sec, 52 deg C.: 30 sec, 68 deg C.: 1 min) and then the final reaction at 68 deg C. for 5 min. The EcoRI site-added EYFP cDNA thus amplified was digested with EcoRI and inserted into the aforementioned pECFP-mouse Epac2 expression vector which had been digested with EcoRI. This vector thus obtained was used as the fluorescent-labeled mouse Epac2 expression vector.

Construction of mutant-type fluorescent-labeled moue Epac2 expression vector—Step 1:

Using the aforementioned mutant-type mouse Epac2 expression vector (pFLAG-Epac2 G114E G422D) as a template, PCR was performed with primer BglII-Epac2 (5′-agatctatggtcgctgcgca-3′: SEQ ID NO:9, aforementioned) and primer Epac2-EcoRI (5′-gaattctggccttcgagg-3′: SEQ ID NO:10, aforementioned) to amplify mutant-type mouse Epac2 cDNA. Using PfuDNA polymerase, the PCR procedure consisted of 10 cycles of (95 deg C.: 30 sec, 55 deg C.: 30 sec, 68 deg C.: 2 min), and 30 cycles of (94 deg C.: 30 sec, 50 deg C.: 30 sec, 68 deg C.: 3 min) and then the final reaction at 68 deg C. for 5 min. The mutant-type mouse Epac2 cDNA thus amplified was digested with BglII and EcoRI, and then inserted into the pECFP-C1 vector (Clontech). This vector thus obtained was named the mutant-type pECFP-mouse Epac2 expression vector.

### Construction of Mutant-Type Fluorescent-Labeled Mouse Epac2 Expression Vector—Step 2:

Using the pEYFP-N1 vector (Clontech) as a template, PCR was performed with primer GFP-fw (5′-atggtgagcaagggcg-3′: SEQ ID NO:15, aforementioned) and primer GFP-ry (5′-cttgtacagctcgtccat-3′: SEQ ID NO:16) to amplify the yellow fluorescent protein EYFP cDNA. Using PfuDNA polymerase, the PCR procedure followed consisted of 10 cycles of (95 deg C.: 30 sec, 55 deg C.: 30 sec, 72 deg C.: 1 min), and 30 cycles of (94 deg C.: 30 sec, 52 deg C.: 30 sec, 68 deg C.: 1 min) then the final reaction at 68 deg C. for 5 min. Then, using the EYFP cDNA thus obtained by PCR as a template, PCR was performed with primer EcoRI-EYFP (5′-gaattcatggtgagcaagg-3′: SEQ ID NO:17, aforementioned) and primer EYFP-EcoRI (5′-gaattccttgtacagctcgt-3′: SEQ ID NO:18, aforementioned) to amplify the EcoRI site-added EYFP cDNA. Using PfuDNA polymerase, the PCR procedure followed consisted of 10 cycles of (95 deg C.: 30 sec, 55 deg C.: 30 sec, 72 deg C.: 1 min), and 30 cycles of (94 deg C.: 30 sec, 52 deg C.: 30 sec, 68 deg C.: 1 min) and then the final reaction at 68 deg C. for 5 min. The EcoRI site-added EYFP cDNA thus amplified was digested with EcoRI, and inserted into the mutant-type pECFP-mouse Epac2 expression vector which had been digested with EcoRI. The vector thus obtained was named fluorescent-labeled mutant-type mouse Epac2 expression vector.

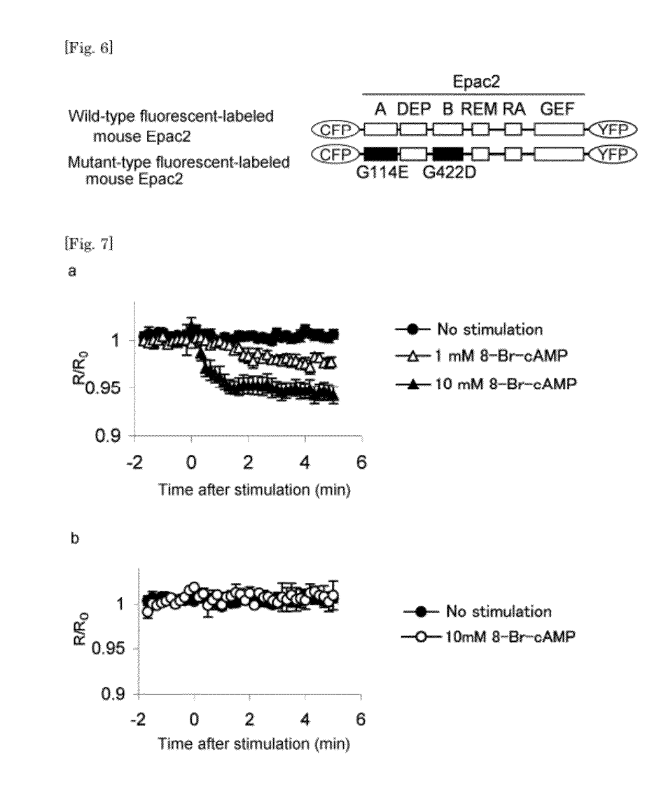

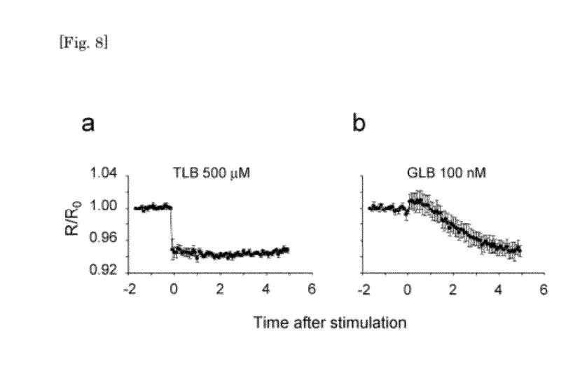

FIG. 6 shows a schematic diagrams of the secondary structure of fluorescent-labeled mouse Epac2 and fluorescent-labeled mutant-type mouse Epac2.

### Cell Culture and Transformation:

Culture of MIN6 and COS-1 cells were conducted in Dulbecco's modified Eagle's medium (DMEM) containing 10% heat-inactivated fetal bovine serum at 5% CO₂. Transformation of the cells was performed using FuGENE6 transfection reagent (Roche Molecular Biochemicals, Basel, Switzerland).

### Observation and Dynamic Analysis of Fluorescent-Labeled Mouse Epac2 by FRET Technique:

Two days before the day for measurement, the cells were transformed with fluorescent-labeled mouse Epac2 expression vector and then cultured. On the day before the day for measurement, the cells were transferred onto the glass culture dishes of 25 mm in diameter, and culture was continued. About 48 hours after the transformation, the culture medium was replaced with HEPES-KRB buffer (HEPES buffer containing 133.4 mM NaCl, 4.7 mM KCl, 1.2 mM KH2PO₄, 1.2 mM MgSO₄, 2.5 mM CaCl₂, 5.0 mM NaHCO₃, 2.8 mM glucose, and 0.2% BSA (pH 7.4)). The cells were observed through a confocal microscope (FV1000, Olympus Corporation) equipped with the UPlanSApo objective lens (100*oil/1.40 NA) to set the focus of the lens on the cells, and excitation light with the wavelength of 440 nm was irradiated using 440 nm LD laser (FV5-LDPSU, Olympus Corporation) at the power of 0.5%. Fluorescent light emitted from thus excited fluorescent-labeled mouse Epac2 was observed through two fluorescence filters, 480DF30 (for 480 nm light) and 535DF25 (for 535 nm light) as dual images of the fluorescent light every 5 seconds for dynamic analysis of the emitted fluorescent light. The results of the dynamic analysis, after first calculating the ratio R in intensity of fluorescence at 535 nm to 480 nm at each measurement (ratio of intensity at 535 nm/intensity at 480 nm), were expressed in the value which was derived by dividing the ratio by the initial value R₀ (ratio of intensity at 535 nm/intensity at 480 nm before addition of an agent), i.e., R/R₀. All the observation was carried out at room temperature.

### Binding Experiment of Sulfonylureas:

COS1 cells were transformed with mouse Epac2 expression vector. Two days after transformation, the cells were washed twice with a buffer (20 mM HEPES containing 119 mM NaCl, 4.7 mM KCl, 2.5 mM CaCl₂, 1.2 mM KH2PO₄, 1.2 mM MgSO₄, 5.0 mM NaHCO₃, (pH 7.4)) and suspended in the same buffer at the density of 2.5-5.2*10⁵ cells/400 micro L. The cells were subdivided into 400 micro L each, and after addition of tritium-labeled glibenclamide, were let stand for one hour at room temperature. Unlabeled glibenclamide or tolbutamide, or the like was added at the same time and was let compete with tritium-labeled glibenclamide. The COS-1 cells were fractured, and tritium-labeled glibenclamide bound to the proteins within the cells was adsorbed onto Whatman GF/C membrane by vacuum filtration (Whatman, Maidstone, UK). The membrane was washed four times with the same buffer which had been ice cooled, and then measured for radioactivity on a liquid scintillation counter.

### GTP-RAP1 Pull-Down Assay:

GTP-RAP1 pull-down assay was performed according to the method described in Shibasaki T. et al. Proc. Natl. Acad. Sci. USA 104, 19333-9 (2007). Namely, MIN6 cells which had been let stand for 30 minutes in HEPES-KRB buffer in advance were cultured for further 15 minutes in HEPES-KRB buffer containing an agent of interest and 2.8 mM glucose. Then, the cells were fractured, and to the cell lysate thus obtained was added glutathione resin which carried GST-Ra1GDS-RID bound to it (SIGMA). After 90 minutes of incubation at 4 deg C., the resin was separated by centrifugation, washed several times with HEPES-KRB buffer, and subjected to SDS-PAGE gel electrophoresis. After the SDS-PAGE gel electrophoresis, Western blotting was carried to transfer the protein onto PVDF membrane, and Rap1 transferred on the membrane was detected with anti-Rap1 antibody (Santa Cruz Biotechnology, Inc., USA).

### Insulin Secretion Experiment:

Mouse pancreatic beta-cells were isolated from wild-type mice (C57BL/6) or Epac2-lacking mice, and cultured for two days. The mouse pancreatic beta-cells were let stand in HEPES-KRB buffer containing 2.8 mM glucose for 30 minutes. The cells then were dispensed to 92-well plates in such a manner that five pancreatic islets of a like size were contained in each well, and cultured for 15 minutes in 100 micro L HEPES-KRB buffer containing an agent of interest and 2.8 mM glucose. The amount of insulin released in the medium and that in the cells were measured using an insulin assay kit (Medical Biological Laboratories Co. Ltd.). The amount of secreted insulin was normalized by the amount of insulin in the cells.

### Oral Glucose Tolerance Test:

Mice fasted for 16 hours were orally administered with 1.5 g/kg body weight of glucose alone or with 1.5 g/kg body weight of glucose plus 100 mg/kg body weight of tolbutamide. Peripheral blood was sampled at a predetermined point of time, and serum insulin concentration and blood glucose concentration were measured with an ELISA kit (mftd. by Morinaga) and Antisense III glucose analysis device (Bayer Yakuhin, Ltd.), respectively.

Observation of the change in three-dimensional structure of fluorescent-labeled mouse Epac2 by FRET technique:

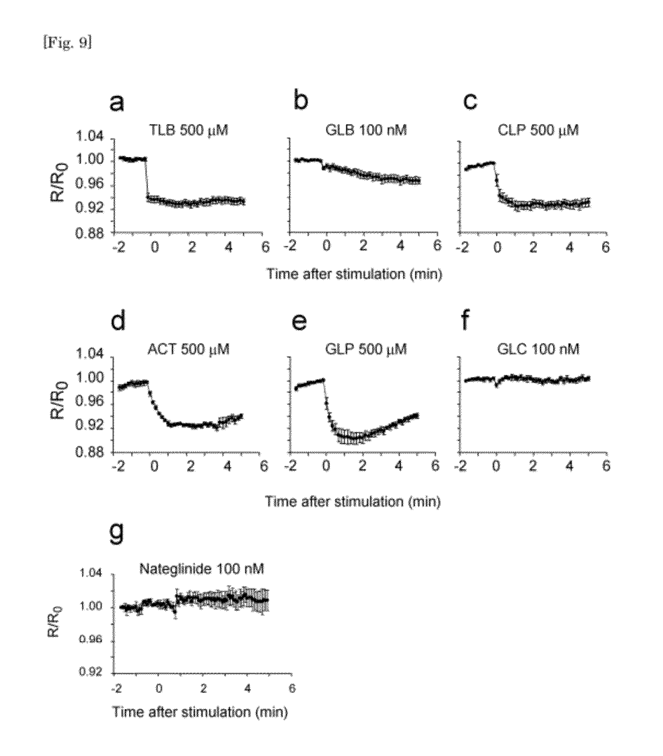

When the COS-1 cells transformed with fluorescent-labeled mouse Epac2 expression vector (fluorescent-labeled mouse Epac2 expressing COS-1 cells) were examined using the FRET technique for 6 minutes in a buffer solution containing 1 mM or 10 mM 8-bromo-cAMP, an analogue to cAMP, a concentration-dependent decrease in R/R₀ value was observed (FIG. 7a). This decrease in R/R₀ value was thought to be the result of a change in FRET due to a change in the three-dimensional structure of the fluorescent-labeled mouse Epac2 which was caused by the binding of 8-bromo-cAMP to it. On the other hand, when the COS-1 cells transformed with the fluorescent-labeled mutant-type mouse Epac2 expression vector, whose cAMP binding site was destroyed, were examined in the same manner, no decrease in R/R₀ value was observed (FIG. 7b). This was thought to be due to 8-bromo-cAMP being unable to bind to the mutant-type fluorescent-labeled mouse Epac2, causing no change in the three-dimensional structure of the mutant-type fluorescent-labeled mouse Epac2, which led to no change in FRET. In addition, when culture was conducted without addition of 8-bromo-cAMP, no change in R/R₀ was observed in either of the cases (FIG. 7a, FIG. 7b).

These results demonstrate that the change in the higher structure of fluorescent-labeled mouse Epac2 caused by the binding of 8-bromo-cAMP to it can be observed within the cells by the FRET technique. Thus, the binding of cAMP to Epac2 is one of the steps of signal transmission via Epac2, and it can be observed over time using cells expressing fluorescent-labeled mouse Epac2.

Binding of SU agents to fluorescent-labeled mouse Epac2:

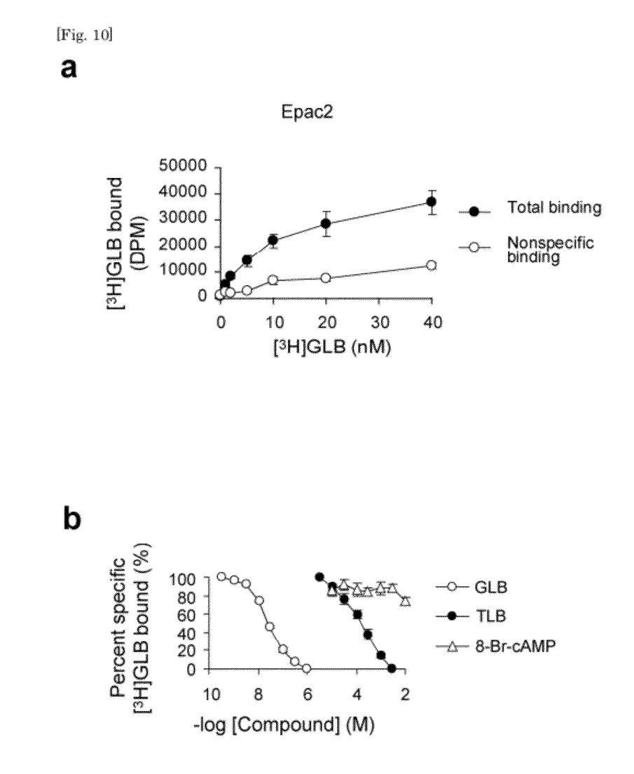

When MIN6 cells which had been transformed with the fluorescent-labeled mouse Epac2 expression vector was examined using the FRET technique in the presence of an SU agent, tolbutamide or glibenclamide, concentration-dependent decrease in R/R₀ value was observed (FIG. 8). As the SU agents are known to exhibit their function via their binding to SUR1, a component of K_{ATP} channels, a possibility couldn't be ruled out that this decrease in R/R₀ value observed here was an indirect effect through SUR1.

Thus, fluorescent-labeled mouse Epac2 expressing COS-1 cells that did not express SUR1 were examined using the FRET technique in the presence of one of various SU agents, and this revealed that a decrease in R/R₀ was observed in the presence of tolbutamide, glibenclamide, chlorpropamide, acetohexamide, and glipizide (FIG. 9a-e). The results suggest that these SU agents act not via SUR1 but directly on Epac2 to alter the higher structure of it. This was surprising because Epac2 had never been expected to be a target of SU agents. On the other hand, no change in R/R₀ value was observed in the presence of gliclazide or nateglinide, although these are among SU agents. This suggests that these agents do not act directly on Epac2 to cause an alteration in its higher structure (FIG. 9f-g). As Epac2 is a signal transmitter which plays a role in insulin secretion, the above results suggest that there are different groups of SU agents with respect to their mechanism of action, one working via Epac2 and the other not.

Then, in order to verify whether SU agents bind directly to Epac2, a sulfonylurea binding experiment was carried out using tritium-labeled glibenclamide. The COS-1 cells transformed with mouse Epac2 expression vector were cultured in the presence of tritium-labeled glibenclamide at a concentration of 1-40 nM, and the cells then were fractured to measure the radioactivity of tritium-labeled glibenclamide bound to intracellular factors of COS-1. The amount of nonspecifically bound tritium-labeled glibenclamide was regarded as the radioactivity measured when cultured in the copresence of non-radioactive-labeled glibenclamide at the concentration of 100 micro M. As a result, the amount of specifically bound tritium-labeled glibenclamide was found increased in a concentration-dependent manner (FIG. 10a). On the other hand, no specific binding was observed with normal COS-1 cells (data not shown). These results show that glibenclamide specifically binds to Epac2.

Next, the COS-1 cells transformed with mouse Epac2 expression vector were cultured in the copresence of unlabeled glibenclamide (GLB), tolbutamide (TLB) or 8-bromo-cAMP (8-Br-cAMP) and 10 nM tritium-labeled glibenclamide to let them compete for binding to Epac2. As a result, IC₅₀ was found to be 25 nM for unlabeled glibenclamide, 240 micro M for tolbutamide, and almost no competition was observed with 8-bromo-cAMP (FIG. 10b). These results suggest that the affinity of glibenclamide for Epac2 is stronger than that of tolbutamide. In addition, they further suggest that the glibenclamide binding site in the Epac2 molecule does not overlap with the cAMP binding site.

These results indicate that it is possible to observe whether or not agents, including SU agents, bind to Epac2, using the FRET technique utilizing fluorescent-labeled Epac2 as a sensor.

### Involvement of Epac2 in Activation of Rap1 by SU Agents:

Epac2 has the GEF activity and activates Rap1 by converting it to GTP-bound Rap1 through its binding to GTP. This reaction constitute part of signal transmission for insulin secretion in beta-cells. Thus, it was examined whether or not Rap1 is activated with SU agents, using MIN6 cells, which originate from beta-cells. Activation of Rap1 was measured by determining GTP-bound Rap1, using GTP-RAP1 pull-down assay. As a positive control 8-bromo-cAMP and TPA, both known as Rap1 activator compounds, were used.

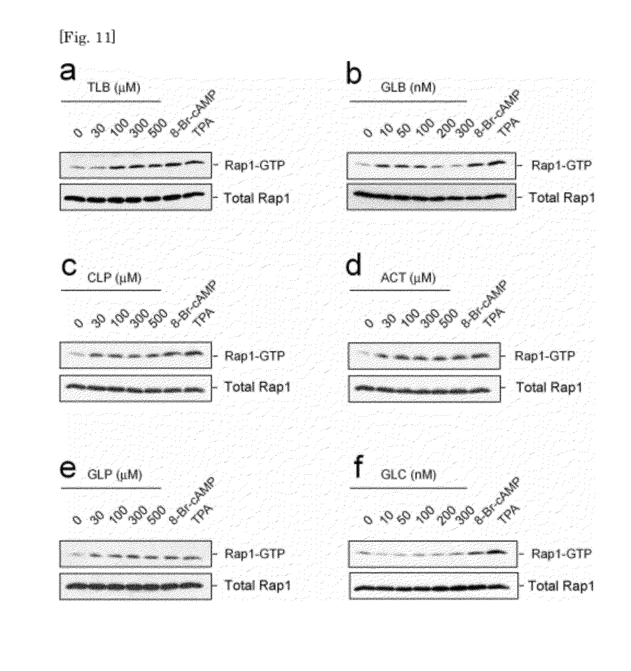

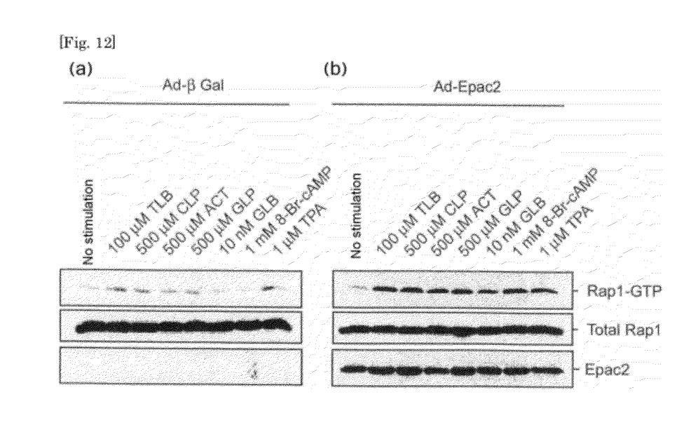

As a result, tolbutamide, glibenclamide, chlorpropamide, acetohexamide, and glipizide were found to increase the amount of GTP-bound Rap1 within the MIN6 cells in a concentration-dependent manner, thus activating Rap1 (FIG. 11a-e). Glibenclamide, however, while up to the concentration of 100 nM, increasing the amount of GTP-bound Rap1, failed to increase it at concentrations higher than that (FIG. 11b). On the other hand, gliclazide was found to have no effect on the amount of GTP-bound Rap1 within the MIN6 cells, thus activating no Rap1 (FIG. 11).

Tolbutamide, glibenclamide, chlorpropamide, acetohexamide, and glipizide, all of which activated Rap1, are SU agents with which decrease in R/R₀ value was observed using the FRET technique, whereas gliclazide, which did not activate Rap1, is an SU agent with which no decrease in R/R₀ value was observed. These results suggest that activation of Rap1 by SU agents takes place via binding of SU agents to Epac2.

Then, using Epac2-lacking pancreatic beta-cells, examination was performed as to whether Rap1 was activated by SU agents. Activation of Rap1 was measured by determining the amount of GTP-bound Rap1 using GTP-RAP1 pull-down assay. As positive controls, 8-bromo-cAMP and TPA were used, which are known to activate Rap1.

As a result, none of tolbutamide, glibenclamide, chlorpropamide, acetohexamide, glipizide, all of which had been found to activate Rap1 in MIN6 cells, activated no Rapt in the Epac2-lacking pancreatic beta-cells (FIG. 12a). However, when similar experiments were conducted after transforming the Epac2-lacking pancreatic beta-cells with the mouse Epac2 expression vector, all of the above agents were found to activate Rap1 (FIG. 12b). These results revealed that it is via Epac2 that those SU agents activate Rap1. On the other hand, nateglinide, which had no influence on R/R₀ values as examined using the FRET technique, did not activate Rap1 even in mouse Epac2 expression vector-transformed Epac2-lacking pancreatic beta-cells (data not shown).

These results indicate that those agents observed to bind Epac2 using the FRET technique employing fluorescent-labeled Epac2 as a sensor, activate Rapt via Epac2. Namely, the results indicate that the FRET technique, employing fluorescent-labeled Epac2 as a sensor, can be used to screen for agents which activate Rap1 via Epac2.

The effect of SU agents on insulin secretion via Epac2:

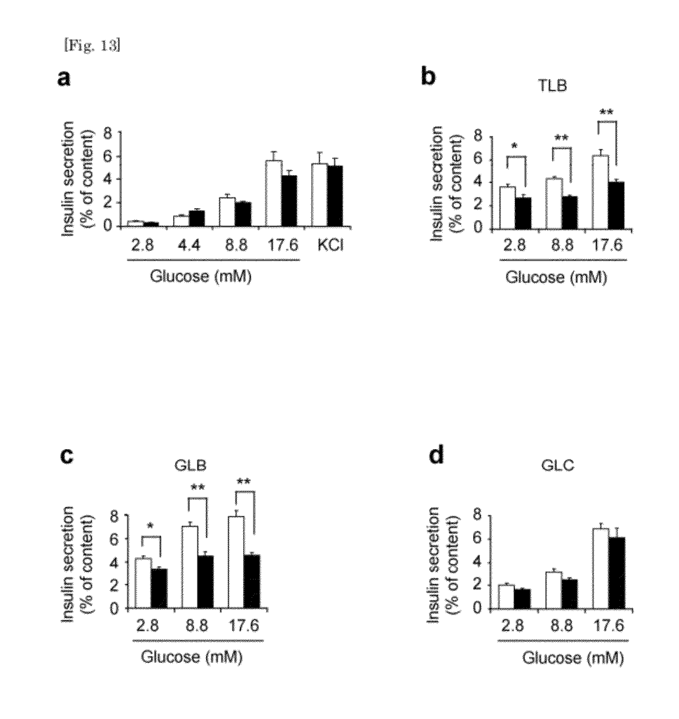

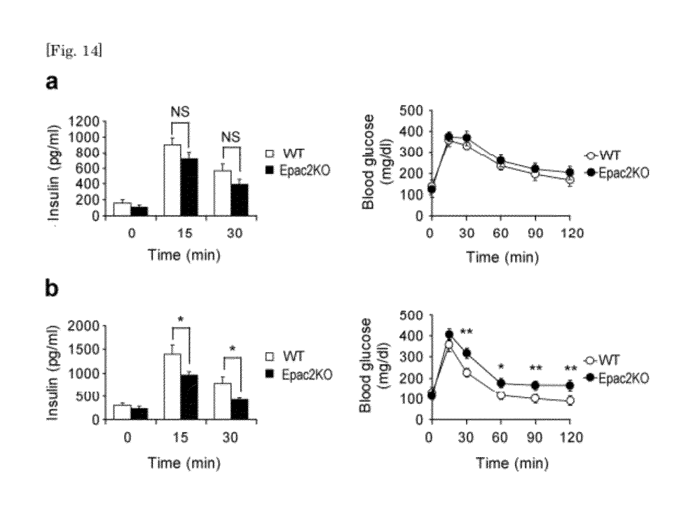

The effect of SU agents on insulin secretion via Epac2 was examined in an insulin secretion experiment using pancreatic islets. First, pancreatic islets obtained from wild-type mice and Epac2-lacking mice were stimulated with glucose, and the amount of secreted insulin was determined. As a result, there was found no significant difference in insulin secretion between the two (FIG. 13a). This was also the case when stimulation was done with KCl (FIG. 13a). Then, pancreatic islets obtained from wild-type mice and Epac2-lacking mice were stimulated with tolbutamide (100 nM) or glibenclamide (10 nM), with which lowered R/R₀ values was detected using the FRET technique, and measured the amount of secreted insulin in the same manner. As a result, after the stimulation of these agents, the pancreatic islets from Epac2-lacking mice showed markedly low insulin secretion compared with the pancreatic islets from wild-type mice (FIG. 13b, c). On the other hand, there was found no significant difference between the two groups with gliclazide (10 nM), with which no change was found in R/R₀ using the FRET technique (FIG. 13d). These results indicate that a pathway through Epac2 is necessary for those SU agents which lower R/R₀ value as determined using the FRET technique to exhibit their full pharmacological effects.

To investigate the effects of SU agents via Epac2 in vivo, the effect of tolbutamide on serum insulin and blood glucose concentrations were examined after oral administration of glucose according to oral glucose tolerance test. First, glucose alone was administered to wild-type mice and Epac2-lacking mice, and their serum insulin and blood glucose concentrations were measured, which gave no significant difference between the two groups (FIG. 14a). Next, glucose was administered together with tolbutamide, and measurement was conducted in the same manner. The result showed that the serum insulin concentration was low in the Epac2-lacking mice compared with the wild-type mice (FIG. 14b). At the same time, corresponding to the serum insulin concentration, it was shown that the blood glucose concentration was high in the Epac2-lacking mice compared with the wild-type mice (FIG. 14b).

The above results indicate that the effects of SU agents via Epac2 are exhibited also in vivo, and that activation of the Epac2/Rap1 signal transmission pathway is necessary for certain SU agents, including tolbutamide, to fully exhibit their effect. Further, it was found that because agents which activate Rap1 via Epac2 can be screened using the FRET technique employing fluorescent-labeled Epac2 as a sensor, the same method can be used to screen for agents which potentiate insulin secretion via Epac2. For example, since SU agents that can be used as agents for treating diabetic patients who lack K_{ATP} channels are limited to those which activate the Epac2/Rap1 signal transmission pathway, such agents can be screened by the method. On the contrary, agents which do not activate the Epac2/Rap1 signal transmission pathway also can be screened.

### INDUSTRIAL APPLICABILITY

The present invention can be utilized as a material to perform screening for insulin secretion-potentiating agents applicable to diabetic patients, and also as a method for such screening.

### [Sequence Listing Free Text]

SEQ ID NO:1=Mouse wild-type Epac2
SEQ ID NO: 3=Human wild-type Epac2
SEQ ID NO: 5=pFLAT-CMV-2 full-length DNA
SEQ ID NO: 6=Multicloning site, pFLAT-CMV-2
SEQ ID NO: 7=Mouse mutant Epac2 (Epac2 G114E G422D)
SEQ ID NO: 9=Primer BglII-Epac2 SEQ ID NO: 10=Primer Epac2-EcoRI
SEQ ID NO: 11=Multicloning site, pECFP-C1

### SEQ ID NO: 12=ECFP

### SEQ ID NO: 13=Synthetic Construct

SEQ ID NO: 14=Multicloning site, pEYFP-N1

### SEQ ID NO: 15=Primer GFP-fw

### SEQ ID NO: 16=Primer GFP-ry

### SEQ ID NO: 17=Primer EcoRI-EYFP

### SEQ ID NO: 18=Primer EYFP-EcoRI

### SEQ ID NO: 19=EYFP

### SEQ ID NO: 20=Synthetic Construct

### [Sequence Listing]

GP128-PCT ST25.txt

1. A DNA encoding fluorescent-labeled Epac2 comprising two different DNAs encoding two different fluorescent proteins which emit fluorescent light with wavelength differing from each other and a DNA encoding Epac2 which are fused together in-frame.

2. The DNA according to claim 1, wherein the two different fluorescent proteins are a cyan fluorescent protein and a yellow fluorescent protein.

3. The DNA according to claim 2, wherein the DNA encoding the cyan fluorescent protein and the DNA encoding the yellow fluorescent protein are fused to the 5′- and 3′-terminuses, respectively, of the DNA encoding Epac2.

4. The DNA according to claim 2, wherein the cyan fluorescent protein is ECFP and the yellow fluorescent protein is EYFP.

5. An expression vector comprising an incorporated DNA according to claim 1.

6. The expression vector according to claim 5, wherein the expression vector is for mammalian cells.

7. A cell which is a transformant carrying the expression vector according to claim 5.

8. The cell according to claim 7, wherein the cell is a mammalian cell.

9. The cell according to claim 8, wherein the cell does not express SUR1 gene.

10. The cell according to claim 9 which is a COS cell.

11. A fluorescent-labeled Epac2 which is a fusion protein comprising Epac2 and two different fluorescent proteins fused thereto, wherein the two different fluorescent proteins emit fluorescent light with wavelength differing from each other.

12. A fluorescent-labeled Epac2 which is a fusion protein comprising two different fluorescent proteins and Epac2, wherein the fusion protein is expressed in the cell according to claim 7.

13. A method of screening for an insulin secretion-potentiating agent comprising the steps of providing candidate compounds for an insulin secretion-potentiating agent, bringing each candidate compound into contact with the cell according to claim 7, irradiating the cell with the excitation light for the shorter wavelength fluorescent protein of the two different fluorescent proteins before and after the contact of the cell with the candidate compound to measure the intensity of the fluorescent light emitted from each of the two different fluorescent proteins included in the fluorescent-labeled Epac2 in the cell, detecting changes in the intensity ratio between the two different fluorescent lights before versus after the contact of the candidate compound with the cell, and selecting a candidate compound which caused a change as an insulin secretion-potentiating agent.

14. A method of screening for an insulin secretion-potentiating agent comprising the steps of providing candidate compounds for an insulin secretion-potentiating agent, bringing each candidate compound into contact with the fluorescent-labeled Epac2 according to claim 11, irradiating the same with the excitation light for the shorter wavelength fluorescent protein of the two different fluorescent proteins before and after the contact of the same with the candidate compound to measure the intensity of the fluorescent light emitted from each of the two different fluorescent proteins included in the fluorescent-labeled Epac2, detecting changes in the intensity ratio between the two different fluorescent lights before versus after the contact with the candidate compound, and selecting a candidate compound which caused a change as an insulin secretion-potentiating agent.


SEQUENCE LISTING (20 sequences)

SEQ ID NO: 1 - DNA, 3036 bp, Mus musculus
  CDS (1)..(3036): Mouse wild-type Epac2
atggtcgctgcgcacgctgcacactctcagtcctcggccgagtggatcgcctgcctggataaaaggccgttggagcgatctagtgaagatgtggacataattttcacgcggctgaaaggagttaaagcttttgagaaatttcacccaaacctccttcgtcagatttgtttatgcggttactatgagaacctggaaaaaggaatcacactgtttcgccaaggggatattggaaccaactggtatgctgtcctggctgggtctttggatgttaaagtgtctgagaccagcagtcaccaggatgcggtgaccatctgcactctgggaattgggacagcctttggagagtccattctggataacacccctcgccatgcaaccatcgttaccagggagagcagcgaacttctccgcattgagcaggaggacttcaaggcactatgggagaaataccgacagtatatggccggacttctggctcctccctatggtgttatggaaacgggctctaacaatgacaggattcctgacaaggagaatacacctctcattgaaccccacgttcctctccgtcctgctcacaccattaccaaggtcccttcagagaagatcctcagagctggaaaaattttacgaattgccattctctctcgagctccccacatgataagagacagaaagtaccacctaaagacatacagacaatgctgtgttgggactgagctggtagactggatgatacagcagacatcctgtgttcactcgcggactcaagctgttggcatgtggcaagtcttgctggaagatggtgtcctcaaccatgtggaccaggagcgccatttccaagacaaatatttattttatcgatttctggatgacgagcgtgaggatgcccctttgcctactgaggaagagaagaaggagtgtgatgaagaacttcaggacaccatgctgctgctctcacagatgggccctgacgcccacatgagaatgatcctgcgaaaaccacctggccagaggactgtggatgacctagagattatctacgacgagctccttcatattaaagccttatcccatctctctaccacagtgaaacgggagttagcaggtgttctcatttttgagtctcacgccaaaggaggaactgtgttgtttaaccagggggaagaaggtacctcctggtacatcattctgaaaggatccgtgaatgtagtcatttatggcaagggtgtggtctgcaccctgcacgaaggagatgactttggcaagttagctctagtgaacgatgctccaagagctgcctccattgttcttcgggaagataattgtcacttcctaagagtcgacaaggaagacttcaatcggattctgagggacgttgaggcgaatacagtcagacttaaagaacatgaccaagatgtcttggtactggagaaggtcccagcagggaacagagctgctaatcaaggaaactcacagcctcagcaaaagtatactgtgatgtcaggaacacctgaaaagattttagagcattttctagaaacaatacgccttgagccatcgttgaatgaagcaacagattcggttttaaatgactttgttatgatgcactgtgtttttatgccaaatacccagctttgccctgcccttgtggcccattaccacgcacagccttctcaaggtaccgagcaggagagaatggattatgccctcaacaacaagaggcgggtcatccgcttggtcctgcagtgggcggccatgtatggcgatctcctccaagaagatgatgtggccatggccttcctggaggagttctatgtgtctgtatcagatgacgcacggatgatggctgccttcaaggagcagctgccagagctggagaagattgtcaagcaaatctcagaagacgcaaaagctccacagaagaagcacaaggtgcttttgcaacagttcaacacaggtgacgagagggcccagaagcgtcagcctattcgtggctctgatgaggttttgttcaaggtctactgcatcgaccacacctatactaccattcgtgtgccggtagctgcctcggtgaaggaagtcatcagtgcagtagctgacaaactgggctcaggggaaggcctgatcatcgtcaagatgaactctggaggagaaaaggtggtgctgaaatctaatgatgtttcagtatttacgacgctcaccattaatggacgcctgtttgcctgcccgagagagcaattcgactcactgactcccttgccggaacaggaaggcccgaccactgggacagtgggaacatttgagctgatgagctcgaaagacctggcgtaccagatgacaacctacgattgggaactcttcaactgtgtgcatgagctggagctaatctaccacacatttggaaggcataattttaaaaagaccacggcaaacttggatttgttcctgaggaggtttaatgaaattcagttttgggttgtcactgaggtctgcctttgttcccagctcagcaaacgtgttcagcttttgaaaaaatttatcaagatagcggctcactgcaaggagtacaaaaatctaaattcctttttcgccatcgtcatgggactcagcaacgtggccgtgagccgcttggcactaacgtgggagaaactgccgagcaagtttaagaagttctatgcggagtttgagagcttgatggatccttccagaaaccacagggcatacaggctgacagcagccaagctggagccccctctcatccctttcatgcccttgcttattaaagatatgacatttactcatgaggggaacaagacgttcattgacaatctagtaaactttgaaaaaatgcgcatgattgcaaacactgccagaacagtacggtactacaggagccagcccttcaatccggatgccgctcaagctaataagaaccatcaggatgtccggagttatgtacggcaattaaatgtgattgacaaccagagaactttatcacagatgtcacacagattagagcctcgaaggccatag

SEQ ID NO: 2 - protein, 1011 aa, Mus musculus
MVAAHAAHSQSSAEWIACLDKRPLERSSEDVDIIFTRLKGVKAFEKFHPNLLRQICLCGYYENLEKGITLFRQGDIGTNWYAVLAGSLDVKVSETSSHQDAVTICTLGIGTAFGESILDNTPRHATIVTRESSELLRIEQEDFKALWEKYRQYMAGLLAPPYGVMETGSNNDRIPDKENTPLIEPHVPLRPAHTITKVPSEKILRAGKILRIAILSRAPHMIRDRKYHLKTYRQCCVGTELVDWMIQQTSCVHSRTQAVGMWQVLLEDGVLNHVDQERHFQDKYLFYRFLDDEREDAPLPTEEEKKECDEELQDTMLLLSQMGPDAHMRMILRKPPGQRTVDDLEIIYDELLHIKALSHLSTTVKRELAGVLIFESHAKGGTVLFNQGEEGTSWYIILKGSVNVVIYGKGVVCTLHEGDDFGKLALVNDAPRAASIVLREDNCHFLRVDKEDFNRILRDVEANTVRLKEHDQDVLVLEKVPAGNRAANQGNSQPQQKYTVMSGTPEKILEHFLETIRLEPSLNEATDSVLNDFVMMHCVFMPNTQLCPALVAHYHAQPSQGTEQERMDYALNNKRRVIRLVLQWAAMYGDLLQEDDVAMAFLEEFYVSVSDDARMMAAFKEQLPELEKIVKQISEDAKAPQKKHKVLLQQFNTGDERAQKRQPIRGSDEVLFKVYCIDHTYTTIRVPVAASVKEVISAVADKLGSGEGLIIVKMNSGGEKVVLKSNDVSVFTTLTINGRLFACPREQFDSLTPLPEQEGPTTGTVGTFELMSSKDLAYQMTTYDWELFNCVHELELIYHTFGRHNFKKTTANLDLFLRRFNEIQFWVVTEVCLCSQLSKRVQLLKKFIKIAAHCKEYKNLNSFFAIVMGLSNVAVSRLALTWEKLPSKFKKFYAEFESLMDPSRNHRAYRLTAAKLEPPLIPFMPLLIKDMTFTHEGNKTFIDNLVNFEKMRMIANTARTVRYYRSQPFNPDAAQANKNHQDVRSYVRQLNVIDNQRTLSQMSHRLEPRRP

SEQ ID NO: 3 - DNA, 3036 bp, Homo sapiens
  CDS (1)..(3036): Human wild-type Epac2
atggtcgctgcgcacgctgcccattcttcctcctctgccgagtggatcgcctgcctggataaaagaccactggagcgatccagcgaagatgtggatataatcttcactcgactgaaagaagttaaagcttttgagaaatttcacccaaatctccttcatcagatttgcttatgtggttattatgagaatctggaaaagggaataacattatttcgccagggtgatattggaacaaactggtatgctgtcctggcagggtctttggatgttaaagtatctgagaccagcagtcaccaggatgctgtgaccatctgtaccctgggaattgggacggcctttggagagtccattctggacaacacaccccgccatgcaaccatcgttaccagggagagcagtgaattgctccgcatcgagcagaaggacttcaaggcactatgggagaaatatcgacagtatatggcaggacttctggctcctccttatggtgttatggaaacgggctctaacaatgacaggattcctgacaaggagaacacacctctcattgaacctcacgttcctcttcgtcctgctaacaccattaccaaggtcccttcagagaagatcctcagagctggaaaaattttacgaaatgccattctctctcgagcacctcacatgataagagatagaaaataccacctaaagacatacagacaatgctgtgtgggaactgaactggtggactggatgatgcagcagacaccatgtgttcactcccggactcaagctgttggcatgtggcaagtcctgttagaagatggtgttctcaaccacgtggaccaggagcaccatttccaagacaaatatttattctatcgatttctggatgatgagcacgaggatgcccctttgcctactgaggaggagaagaaggagtgtgatgaggagctccaggacaccatgctgctgctgtcacagatgggccccgacgcccacatgaggatgatccttcgcaaaccacctggccagaggactgtggatgacctagagattatctatgaggagcttcttcatattaaagccttatcccatctttctaccacagtgaaacgagagttagcaggtgttctcatttttgagtctcacgccaaaggagggactgtgttgtttaaccagggggaagaaggtacctcctggtacattattctaaaaggatcagtgaatgtagtcatttacggcaagggtgtggtctgcaccctgcatgaaggagatgacttcggcaagttagcactagtgaatgatgccccacgagctgcctctatcgtcttacgagaagataactgccatttcttaagagtagacaaggaggatttcaaccggatcctaagggacgtggaggcgaatacagtcagacttaaagaacatgaccaagatgtcttggtgctggagaaggtcccagcagggaacagagcttctaatcaaggaaactcacagcctcagcaaaagtatactgtgatgtcaggaacacctgaaaaaattttagagcattttctagaaacaatacgccttgaggcaactttaaatgaagcaacagattctgttttaaatgactttattatgatgcactgtgtttttatgccaaatacccagctttgcccggcactggtggcccactaccacgcacagccttcacaaggtacagaacaggagaaaatggattatgccctcaacaataagaggcgagtcatccgcctggttctacagtgggctgccatgtatggagacctcctgcaagaggatgacgtgtctatggccttcctggaggagttttatgtatctgtatcagatgatgcccggatgattgctgccctcaaggagcaactgccagagttggagaagattgtcaagcaaatctcagaagatgcaaaggcaccacaaaagaagcacaaggttcttttgcaacagttcaatacgggcgatgagagagcccagaagcgccagcctatccgcggctctgatgaagttctgtttaaggtctattgcatggaccacacctacacaaccattcgggtgccagtggccacttcggtgaaggaagtcatcagtgcagttgccgacaagctgggctccggggagggcctgatcatagtcaagatgagttccggaggagaaaaggtggtgctcaaacctaatgatgtttcagtatttacgacgctcaccattaatggacgcctgtttgcttgcccgcgagagcaattcgattcactgactcccttaccagaacaggaaggcccaactgttggaacagtgggaacttttgaactgatgagctccaaagatttagcataccagatgacaatttatgattgggaactcttcaactgcgtgcatgagctggagctaatctatcacacatttggaaggcataattttaaaaagaccacagcaaacttggatttgttcctgaggagatttaatgaaattcagttttgggtcgtcactgagatctgcctttgttctcagctcagcaagcgtgttcagctattaaaaaaatttattaagatagcagcccactgtaaggagtataaaaatctgaattccttttttgccatcgtcatgggactaagtaacgttgctgtgagccgcttggcactaacgtgggagaaactgccaagcaagttcaagaagttctatgcggagtttgaaagtttaatggacccttcaaggaaccacagggcctacaggctgacagtagctaagctggaacctcctctcatccccttcatgcctttgctcattaaagatatgacatttactcatgaggggaacaagacgttcattgacaatctagtaaactttgaaaaaatgcgcatgattgcaaatacggccagaacagtgagatactacaggagccaacccttcaatcctgatgcagctcaagctaataagaaccatcaggatgtccggagttatgtacggcaattaaatgtgattgacaaccagagaactttatcacagatgtcacacagattagagcctcgtcgaccatag

SEQ ID NO: 4 - protein, 1011 aa, Homo sapiens
MVAAHAAHSSSSAEWIACLDKRPLERSSEDVDIIFTRLKEVKAFEKFHPNLLHQICLCGYYENLEKGITLFRQGDIGTNWYAVLAGSLDVKVSETSSHQDAVTICTLGIGTAFGESILDNTPRHATIVTRESSELLRIEQKDFKALWEKYRQYMAGLLAPPYGVMETGSNNDRIPDKENTPLIEPHVPLRPANTITKVPSEKILRAGKILRNAILSRAPHMIRDRKYHLKTYRQCCVGTELVDWMMQQTPCVHSRTQAVGMWQVLLEDGVLNHVDQEHHFQDKYLFYRFLDDEHEDAPLPTEEEKKECDEELQDTMLLLSQMGPDAHMRMILRKPPGQRTVDDLEIIYEELLHIKALSHLSTTVKRELAGVLIFESHAKGGTVLFNQGEEGTSWYIILKGSVNVVIYGKGVVCTLHEGDDFGKLALVNDAPRAASIVLREDNCHFLRVDKEDFNRILRDVEANTVRLKEHDQDVLVLEKVPAGNRASNQGNSQPQQKYTVMSGTPEKILEHFLETIRLEATLNEATDSVLNDFIMMHCVFMPNTQLCPALVAHYHAQPSQGTEQEKMDYALNNKRRVIRLVLQWAAMYGDLLQEDDVSMAFLEEFYVSVSDDARMIAALKEQLPELEKIVKQISEDAKAPQKKHKVLLQQFNTGDERAQKRQPIRGSDEVLFKVYCMDHTYTTIRVPVATSVKEVISAVADKLGSGEGLIIVKMSSGGEKVVLKPNDVSVFTTLTINGRLFACPREQFDSLTPLPEQEGPTVGTVGTFELMSSKDLAYQMTIYDWELFNCVHELELIYHTFGRHNFKKTTANLDLFLRRFNEIQFWVVTEICLCSQLSKRVQLLKKFIKIAAHCKEYKNLNSFFAIVMGLSNVAVSRLALTWEKLPSKFKKFYAEFESLMDPSRNHRAYRLTVAKLEPPLIPFMPLLIKDMTFTHEGNKTFIDNLVNFEKMRMIANTARTVRYYRSQPFNPDAAQANKNHQDVRSYVRQLNVIDNQRTLSQMSHRLEPRRP

SEQ ID NO: 5 - DNA, 4679 bp, Artificial
  pFLAT-CMV-2 full-length DNA
ccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattgatctaccatggactacaaagacgatgacgacaagcttgcggccgcgaattcatcgatagatctgatatcggtaccagtcgactctagaggatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaattccctatagtgagtcgtattaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaattt

SEQ ID NO: 6 - DNA, 91 bp, Artificial
  Multicloning site, pFLAT-CMV-2
atggactacaaagacgatgacgacaagcttgcggccgcgaattcatcgatagatctgatatcggtaccagtcgactctagaggatcccggg

SEQ ID NO: 7 - DNA, 3036 bp, Mus musculus
  CDS (1)..(3036): Mouse mutant Epac2 (Epac2 G114E G422D)
atggtcgctgcgcacgctgcacactctcagtcctcggccgagtggatcgcctgcctggataaaaggccgttggagcgatctagtgaagatgtggacataattttcacgcggctgaaaggagttaaagcttttgagaaatttcacccaaacctccttcgtcagatttgtttatgcggttactatgagaacctggaaaaaggaatcacactgtttcgccaaggggatattggaaccaactggtatgctgtcctggctgggtctttggatgttaaagtgtctgagaccagcagtcaccaggatgcggtgaccatctgcactctgggaattgggacagcctttgaagagtccattctggataacacccctcgccatgcaaccatcgttaccagggagagcagcgaacttctccgcattgagcaggaggacttcaaggcactatgggagaaataccgacagtatatggccggacttctggctcctccctatggtgttatggaaacgggctctaacaatgacaggattcctgacaaggagaatacacctctcattgaaccccacgttcctctccgtcctgctcacaccattaccaaggtcccttcagagaagatcctcagagctggaaaaattttacgaattgccattctctctcgagctccccacatgataagagacagaaagtaccacctaaagacatacagacaatgctgtgttgggactgagctggtagactggatgatacagcagacatcctgtgttcactcgcggactcaagctgttggcatgtggcaagtcttgctggaagatggtgtcctcaaccatgtggaccaggagcgccatttccaagacaaatatttattttatcgatttctggatgacgagcgtgaggatgcccctttgcctactgaggaagagaagaaggagtgtgatgaagaacttcaggacaccatgctgctgctctcacagatgggccctgacgcccacatgagaatgatcctgcgaaaaccacctggccagaggactgtggatgacctagagattatctacgacgagctccttcatattaaagccttatcccatctctctaccacagtgaaacgggagttagcaggtgttctcatttttgagtctcacgccaaaggaggaactgtgttgtttaaccagggggaagaaggtacctcctggtacatcattctgaaaggatccgtgaatgtagtcatttatggcaagggtgtggtctgcaccctgcacgaaggagatgactttgacaagttagctctagtgaacgatgctccaagagctgcctccattgttcttcgggaagataattgtcacttcctaagagtcgacaaggaagacttcaatcggattctgagggacgttgaggcgaatacagtcagacttaaagaacatgaccaagatgtcttggtactggagaaggtcccagcagggaacagagctgctaatcaaggaaactcacagcctcagcaaaagtatactgtgatgtcaggaacacctgaaaagattttagagcattttctagaaacaatacgccttgagccatcgttgaatgaagcaacagattcggttttaaatgactttgttatgatgcactgtgtttttatgccaaatacccagctttgccctgcccttgtggcccattaccacgcacagccttctcaaggtaccgagcaggagagaatggattatgccctcaacaacaagaggcgggtcatccgcttggtcctgcagtgggcggccatgtatggcgatctcctccaagaagatgatgtggccatggccttcctggaggagttctatgtgtctgtatcagatgacgcacggatgatggctgccttcaaggagcagctgccagagctggagaagattgtcaagcaaatctcagaagacgcaaaagctccacagaagaagcacaaggtgcttttgcaacagttcaacacaggtgacgagagggcccagaagcgtcagcctattcgtggctctgatgaggttttgttcaaggtctactgcatcgaccacacctatactaccattcgtgtgccggtagctgcctcggtgaaggaagtcatcagtgcagtagctgacaaactgggctcaggggaaggcctgatcatcgtcaagatgaactctggaggagaaaaggtggtgctgaaatctaatgatgtttcagtatttacgacgctcaccattaatggacgcctgtttgcctgcccgagagagcaattcgactcactgactcccttgccggaacaggaaggcccgaccactgggacagtgggaacatttgagctgatgagctcgaaagacctggcgtaccagatgacaacctacgattgggaactcttcaactgtgtgcatgagctggagctaatctaccacacatttggaaggcataattttaaaaagaccacggcaaacttggatttgttcctgaggaggtttaatgaaattcagttttgggttgtcactgaggtctgcctttgttcccagctcagcaaacgtgttcagcttttgaaaaaatttatcaagatagcggctcactgcaaggagtacaaaaatctaaattcctttttcgccatcgtcatgggactcagcaacgtggccgtgagccgcttggcactaacgtgggagaaactgccgagcaagtttaagaagttctatgcggagtttgagagcttgatggatccttccagaaaccacagggcatacaggctgacagcagccaagctggagccccctctcatccctttcatgcccttgcttattaaagatatgacatttactcatgaggggaacaagacgttcattgacaatctagtaaactttgaaaaaatgcgcatgattgcaaacactgccagaacagtacggtactacaggagccagcccttcaatccggatgccgctcaagctaataagaaccatcaggatgtccggagttatgtacggcaattaaatgtgattgacaaccagagaactttatcacagatgtcacacagattagagcctcgaaggccatag

SEQ ID NO: 8 - protein, 1011 aa, Mus musculus
MVAAHAAHSQSSAEWIACLDKRPLERSSEDVDIIFTRLKGVKAFEKFHPNLLRQICLCGYYENLEKGITLFRQGDIGTNWYAVLAGSLDVKVSETSSHQDAVTICTLGIGTAFEESILDNTPRHATIVTRESSELLRIEQEDFKALWEKYRQYMAGLLAPPYGVMETGSNNDRIPDKENTPLIEPHVPLRPAHTITKVPSEKILRAGKILRIAILSRAPHMIRDRKYHLKTYRQCCVGTELVDWMIQQTSCVHSRTQAVGMWQVLLEDGVLNHVDQERHFQDKYLFYRFLDDEREDAPLPTEEEKKECDEELQDTMLLLSQMGPDAHMRMILRKPPGQRTVDDLEIIYDELLHIKALSHLSTTVKRELAGVLIFESHAKGGTVLFNQGEEGTSWYIILKGSVNVVIYGKGVVCTLHEGDDFDKLALVNDAPRAASIVLREDNCHFLRVDKEDFNRILRDVEANTVRLKEHDQDVLVLEKVPAGNRAANQGNSQPQQKYTVMSGTPEKILEHFLETIRLEPSLNEATDSVLNDFVMMHCVFMPNTQLCPALVAHYHAQPSQGTEQERMDYALNNKRRVIRLVLQWAAMYGDLLQEDDVAMAFLEEFYVSVSDDARMMAAFKEQLPELEKIVKQISEDAKAPQKKHKVLLQQFNTGDERAQKRQPIRGSDEVLFKVYCIDHTYTTIRVPVAASVKEVISAVADKLGSGEGLIIVKMNSGGEKVVLKSNDVSVFTTLTINGRLFACPREQFDSLTPLPEQEGPTTGTVGTFELMSSKDLAYQMTTYDWELFNCVHELELIYHTFGRHNFKKTTANLDLFLRRFNEIQFWVVTEVCLCSQLSKRVQLLKKFIKIAAHCKEYKNLNSFFAIVMGLSNVAVSRLALTWEKLPSKFKKFYAEFESLMDPSRNHRAYRLTAAKLEPPLIPFMPLLIKDMTFTHEGNKTFIDNLVNFEKMRMIANTARTVRYYRSQPFNPDAAQANKNHQDVRSYVRQLNVIDNQRTLSQMSHRLEPRRP

SEQ ID NO: 9 - DNA, 20 bp, Artificial
  Primer BglII-Epac2
agatctatggtcgctgcgca

SEQ ID NO: 10 - DNA, 18 bp, Artificial
  Primer Epac2-EcoRI
gaattctggccttcgagg

SEQ ID NO: 11 - DNA, 94 bp, Artificial
  Multicloning site, pECFP-C1
tacaagtccggactcagatctcgagctcaagcttcgaattctgcagtcgacggtaccgcgggcccgggatccaccggatctagataactgatca

SEQ ID NO: 12 - DNA, 720 bp, Artificial
  ECFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 13 - protein, 239 aa, Artificial
  Synthetic Construct
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 14 - DNA, 94 bp, Artificial
  Multicloning site, pEYFP-N1
gctagcgctaccggactcagatctcgagctcaagcttcgaattctgcagtcgacggtaccgcgggcccgggatccaccggtcgccaccatggtg

SEQ ID NO: 15 - DNA, 16 bp, Artificial
  Primer GFP-fw
atggtgagcaagggcg

SEQ ID NO: 16 - DNA, 18 bp, Artificial
  Primer GFP-rv
cttgtacagctcgtccat

SEQ ID NO: 17 - DNA, 19 bp, Artificial
  Primer EcoRI-EYFP
gaattcatggtgagcaagg

SEQ ID NO: 18 - DNA, 20 bp, Artificial
  Primer EYFP-EcoRI
gaattccttgtacagctcgt

SEQ ID NO: 19 - DNA, 720 bp, Artificial
  EYFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtga

SEQ ID NO: 20 - protein, 239 aa, Artificial
  Synthetic Construct
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK